(19) United States
(12) Patent Application Publication
(10) Pub. No.: US 2004/0152713 A1
(43) Pub. Date: Aug. 5, 2004

(54) Methods for treating joint pain or improving sleep using an estrogen agonist/antagonist

(75) Inventor: Charles D. Petrie, Cranston, RI (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/761,672

(22) Filed: Jan. 21, 2004

Related U.S. Application Data
(60) Provisional application No. 60/441,830, filed on Jan. 22, 2003.

(51) Int. Cl. A61K 31/517; A61K 31/4709

(52) U.S. Cl. 514/266.2; 514/310; 514/314; 514/256

(57) ABSTRACT

The present invention provides methods, pharmaceutical compositions and kits for treating joint pain and/or improving sleep using a SERM of formula (I):

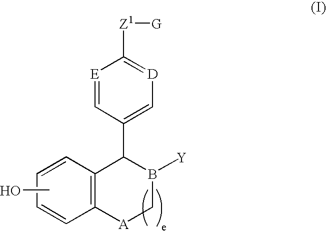

wherein the variables A, B, D, E, e, G, Y and Z¹ are as defined in the specification.

### FIELD OF THE INVENTION

This invention relates to methods for treating joint pain and/or improving sleep using an estrogen agonist/antagonist also known as selective estrogen receptor modulators (hereinafter referred to as SERMs). The invention also relates to pharmaceutical compositions useful for improving sleep.

### BACKGROUND OF THE INVENTION

Almost all persons by age forty have some pathological change in weight bearing joints. Men and women are equally affected, but onset is earlier in men. Joint cartilage, also called hyaline cartilage, is made up of 95% water and extracellular matrix and 5% chondrocytes. The extracellular matrix comprises proteoglycans and Type II collagen. Joint pain can result from various progressive diseases as well as various acute conditions related to the joints. Typical symptomatic treatment of joint pain includes the management of pain by administration of medicaments and changes in lifestyle such as diet and exercise.

Examples of compounds that have been used to treat joint pain include nonsteroidal anti-inflammatory drugs (NSAIDs), such as aspirin, acetominophen, ibuprofen, naproxen, ketoprofen, nabumetone, etodolac, salsalate, sulindac, diclofenac, tolmetin, flurbiprofen, piroxicam, fenoprofen, indomethacin, meclofenamate, oxaprozin, diflunisal and ketorolac; and selective cyclooxygenase-2 (COX-2) inhibitors such as 4-[5-(4-methylphenyl)-3-(trifluoromethyl)-1H-pyrazol-1-yl]benzenesulfonamide (celecoxib), 4-[4-(methylsulfonyl)phenyl]-3-phenyl-2(5H)-furanone (rofecoxib), valdecoxib and etoricoxib. NSAIDs can have unwanted side effects such as ulcers, therefore NSAIDs are sometimes administered with other compounds that ameliorate the side effects of the NSAIDs. Typical compounds that are used in combination with NSAIDs include proton pump inhibitors such as omeprazole; antacids such as sucralfate; and H2 blockers such as ranitidine, cimetidine, famotidine, and nizatidine. In addition, products derived from natural substances have been used to treat various types of joint pain. Examples of natural substances include hyaluronic acid, glucosamine, chondroitin sulfate and capsaicin. Intraarticular corticosteriods have also been used to treat joint pain.

The use of SERMs, such as those of formula I, for the treatment of osteoarthritis has been disclosed in U.S. patent application Publication No. 2001-0041718A1. The use of SERMs, such as those of formula I, for the treatment of rheumatoid arthritis has been disclosed in U.S. patent application Publication No. 2002-0049198A1. There is a continuing need for new methods of treating joint pain in patients suffering from or at risk of suffering from joint pain.

Sleep is a complex process with many parts of the nervous system being involved in controlling it and influencing its different stages. The stages or levels of sleep include drowsiness, light sleep, deep sleep and dream sleep. Individuals vary widely in their requirements for sleep, which is influenced by a variety of factors including the individual's current emotional state. The natural aging process is associated with changes to an individual's circadian and diurnal rhythms. With increasing age the total amount of sleep tends to shorten and the amount of deep sleep can decrease or disappear. Sleep may become more fragmented and interrupted for the elderly and these changes with timing and structure of sleep are often associated with significant morbidity. Similarly, non-elderly individuals may also exhibit disturbances in the normal sleep process. These disturbances in the normal sleep process have been correlated with more frequent napping, decreased daytime alertness and declining intellectual function and cognitive ability.

For individuals suffering from a sleep disorder the normal sleep cycle is also disrupted. The sleep disorder generally affects the afflicted individual's ability to fall and/or stay asleep, and involve sleeping too little, sleeping too much or resulting in abnormal behavior associated with sleep. There are numerous types of sleep disorders with the International Classification of Sleep Disorders having over seventy sleep disorders listed. Sleep disorders, such as various types of dyssomnias and parasomnias, can lead to a lowered quality of life and reduced personal health for those afflicted with them. Sleep disorders can also endanger public safety by contributing to traffic and industrial accidents. In certain instances the sleep disorder can be life threatening.

Examples of current drugs used to improve sleep include those that act as hypnotic sedatives and that may also act as anxiolytics. Benzodiazepines, which act as anxiolytics and are also useful for inducing sleep, enhance the effect of the inhibitory neurotransmitter gamma-aminobutyric acid (GABA). Benzodiazepines, such as diazepam (Valium®) and temazepam (Restoril®), are used as sleeping pills. Triazolam and estazolam are also used to induce sleep. Zolpidem, including its tartrate salt, is used to induce sleep and acts by a mechanism similar to the benzodiazepines. Over the counter remedies used to induce sleep include preparations containing antihistamines, such as diphenhydramine. Sedative antidepressants such as amitryptyline and trazodone have also been used as sleep inducing agents. Dopaminergic agents such as levodopa/carbidopa, bromocriptine mesylate and pergolide, and opiods such as codeine, propoxyphene, oxycodone, pentazocrine, hydrocodone and methadone have also been used as sleep inducing agents. The known therapeutic regimens suffer from numerous problems, including residual effects in daytime function, impairment of memory, potential for addiction or rebound insomnia which may be associated with increased dream intensity and the occurrence of nightmares and the like. There is a continuing need for new methods of improving sleep in patients in need thereof.

It has been found that the SERMs of Formula (I), described hereinafter, are useful in the treatment of joint pain and/or in improving sleep. The present invention provides methods of treating or preventing joint pain and/or improving sleep using a SERM of Formula (I).

### SUMMARY OF THE INVENTION

The present invention provides methods of treating joint pain and/or improving sleep, the methods comprising administering to a patient in need thereof, a therapeutically effective amount of a SERM. A first embodiment of the present invention is the method of treating joint pain and/or improving sleep, the method comprising administering to a patient in need thereof, a therapeutically effective amount of a SERM of formula (I):

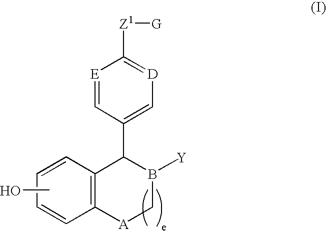

wherein:

A is selected from CH₂ and NR;

B, D and E are independently selected from CH and N;

Y is

(a) phenyl, optionally substituted with 1-3 substituents independently selected from R⁴;

(b) naphthyl, optionally substituted with 1-3 substituents independently selected from R⁴;

(c) C₃-C₈ cycloalkyl, optionally substituted with 1-2 substituents independently selected from R⁴;

(d) C₃-C₈ cycloalkenyl, optionally substituted with 1-2 substituents independently selected from R⁴;

(e) a five membered heterocycle containing up to two heteroatoms selected from the group consisting of —O—, —NR²— and —S(O)ₙ—, optionally substituted with 1-3 substituents independently selected from R⁴;

(f) a six membered heterocycle containing up to two heteroatoms selected from the group consisting of —O—, —NR²— and —S(O)ₙ— optionally substituted with 1-3 substituents independently selected from R⁴; or

(g) a bicyclic ring system consisting of a five or six membered heterocyclic ring fused to a phenyl ring, said heterocyclic ring containing up to two. heteroatoms selected from the group consisting of —O—, —NR²— and —S(O)ₙ—, optionally substituted with 1-3 substituents independently selected from R⁴;

Z¹ is

(a) —(CH₂)ₚ W(CH₂)_{q}—;

(b) —O(CH₂)ₚ CR⁵R⁶—;

(c) —O(CH₂)ₚW(CH₂)_{q}—;

(d) —OCHR²CHR³—; or

(e) —SCHR²CHR³—;

G is

(a) —NR⁷R⁸;

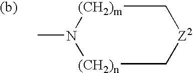

wherein n is 0, 1 or 2; m is 1, 2 or 3; Z² is —NH—, —O—, —S—, or —CH₂—; optionally fused on adjacent carbon atoms with one or two phenyl rings and, optionally independently substituted on carbon with one to three substituents and, optionally, independently on nitrogen with a chemically suitable substituent selected from R⁴; or

(c) a bicyclic amine containing five to twelve carbon atoms, either bridged or fused and optionally substituted with 1-3 substituents independently selected from R⁴; or

Z¹ and G in combination may be

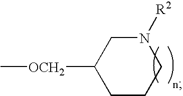

W is

(a) —CH₂—;

(b) —CH═CH—;

(c) —O—;

(d) —NR²—;

(e) —S(O)ₙ—;

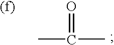

(g) —CR²(OH)—;

(h) —CONR²—;

(i) —NR²CO—;

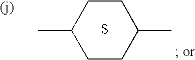

(k) —C≡C—;

R is hydrogen or C₁-C₆ alkyl;

R² and R³ are independently

(a) hydrogen; or

(b) C₁-C₄ alkyl;

R⁴ is

(a) hydrogen;

(b) halogen;

(c) C₁-C₆ alkyl;

(d) C₁-C₄ alkoxy;

(e) C₁-C₄ acyloxy;

(f) C₁-C₄ alkylthio;

(g) C₁-C₄ alkylsulfinyl;

(h) C₁-C₄ alkylsulfonyl;

(i) hydroxy (C₁-C₄)alkyl;

(j) aryl (C₁-C₄)alkyl;

(k) —CO₂H;

(l) —CN;

(m) —CONHOR;

(n) —SO₂NHR;

(o) —NH₂;

(p) C₁-C₄ alkylamino;

(q) C₁-C₄ dialkylamino;

(r) —NHSO₂R;

(s) —NO₂;

(t) -aryl; or

(u) —OH;

R⁵ and R⁵ are independently C₁-C₈ alkyl or together form a C₃-C₁₀ carbocyclic ring;

R⁷ and R⁸ are independently

(a) phenyl;

(b) a C₃-C₁₀ carbocyclic ring, saturated or unsaturated;

(c) a C₃-C₁₀ heterocyclic ring containing up to two heteroatoms, selected from —O—, —N— and —S—;

(d) H;

(e) C₁-C₆alkyl; or

(f) form a 3 to 8 membered nitrogen containing ring with R⁵ or R⁶;

R⁷ and R⁸ in either linear or ring form may optionally be substituted with up to three substituents independently selected from C₁-C₆ alkyl, halogen, alkoxy, hydroxy and carboxy;

a ring formed by R⁷ and R⁸ may be optionally fused to a phenyl ring;

e is 0, 1 or 2;

m is 1, 2 or 3;

n is 0, 1 or 2;

p is 0, 1, 2 or 3;

q is 0, 1, 2 or 3;

or an optical or geometric isomer thereof; or a pharmaceutically acceptable salt, N-oxide, ester, quaternary ammonium salt or prodrug thereof.

A second embodiment of the present invention is the method of treating joint pain and/or improving sleep, the method comprising administering to a patient in need thereof, a therapeutically effective amount of a SERM of formula (IA)

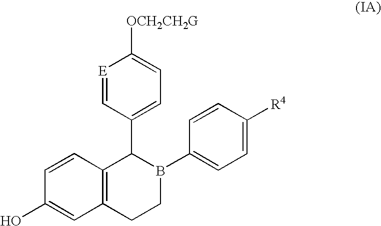

wherein G is

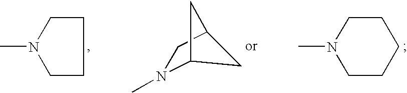

R⁴ is H, OH, F, or Cl; and B and E are independently selected from CH and N or an optical or geometric isomer thereof; or a pharmaceutically acceptable salt, N-oxide, ester, quaternary ammonium salt, or a prodrug thereof.

A third embodiment of the present invention is the method of treating joint pain and/or improving sleep, the method comprising administering to a patient in need thereof, a therapeutically effective amount of (−)-cis-6-phenyl-5-[4-(2-pyrrolidin-1-yl-ethoxy)-phenyl]-5,6,7,8-tetrahydro-naphthalene-2-ol or an optical or geometric isomer thereof; a pharmaceutically acceptable salt, N-oxide, ester, quaternary ammonium salt, or a prodrug thereof.

A fourth embodiment of the present invention is the method of treating joint pain and/or improving sleep, the method comprising administering to a patient in need thereof, a therapeutically effective amount of the D-tartrate salt of (−)-cis-6-phenyl-5-[4-(2-pyrrolidin-1-yl-ethoxy)-phenyl]-5,6,7,8-tetrahydro-naphthalene-2-ol.

Another embodiment of the present invention is the method of treating joint pain, the method comprising administering to a patient in need thereof a therapeutically effective amount of a first compound and a therapeutically effective amount of a second compound. The first compound is a SERM as described in any of the first through fourth embodiments above. The second compound is selected from the group consisting of acetominophen, aspirin, ibuprofen, naproxen, ketoprofen, nabumetone, etodolac, salsalate, sulindac, diclofenac, tolmetin, flurbiprofen, piroxicam, fenoprofen, indomethacin, meclofenamate, oxaprozin, diflunisal, ketorolac, celecoxib, rofecoxib, valdecoxib, etoricoxib, hyaluronic acid, glucosamine, chondroitin and capsaicin. The joint pain referred to in the present methods is not joint pain resulting from osteoarthritis or rheumatoid arthritis. A preferred first compound in the combination method of treating joint pain is (−)-cis-6-phenyl-5-[4-(2-pyrrolidin-1-yl-ethoxy)-phenyl]-5,6,7,8-tetrahydro-naphthalene-2-ol, D-tartrate salt. Preferred second compounds in the combination method of treating joint pain include celecoxib, rofecoxib, valdecoxib and etoricoxib.

Another embodiment of the present invention is the method of improving sleep, the method comprising administering a therapeutically effective amount of a first compound and a therapeutically effective amount of a second compound. The first compound is a SERM as described in any of the first through fourth embodiments above. The second compound is selected from the group consisting of adinazolam, allobarbital, alonimid, alprazolam, amitryptiline, amobarbital, amoxapine, bentazepam, benzoctamine, bromocriptine, brotizolam, bupropion, buspirone, butabarbital, butalbital, capuride, carbocloral, chloral betaine, chloral hydrate, chlordiazepoxide, clomipramine, cloperidone, clorazepate, clorethate, clozapine, codeine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydrocodone, hydroxyzine, imipramine, lithium, lorazepam, lormetazepam, maprotaline, mecloqualone, melatonin, mephobarbital, meprobamate, methadone, methaqualone, midaflur, midazolam, nefazodone, nisobamate, nitrazepam, nortriptyline, oxazepam, oxycodone, paraldehyde, paroxetine, pentazocrine, pentobarbital, pergolide, perlapine, perphenazine, pheneizine, phenobarbital, prazepam, promethazine, propofol, propoxyphene, protryptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem and pharmaceutically acceptable salts thereof. A preferred first compound in the combination method of improving sleep is (−)-cis-6-phenyl-5-[4-(2-pyrrolidin-1-yl-ethoxy)-phenyl]-5,6,7,8-tetrahydro-naphthalene-2-ol, D-tartrate salt. A preferred second compound in the combination method of improving sleep is sertraline and its hydrochloride salt.

Another embodiment of the present invention is a pharmaceutical composition comprising (−)-cis-6-phenyl-5-[4-(2-pyrrolidin-1-yl-ethoxy)-phenyl]-5,6,7,8-tetrahydro-naphthalene-2-ol or a pharmaceutically acceptable salt thereof; and a second compound selected from the group consisting of adinazolam, allobarbital, alonimid, alprazolam, amitryptiline, amobarbital, amoxapine, bentazepam, benzoctamine, bromocriptine, brotizolam, bupropion, buspirone, butabarbital, butalbital, capuride, carbocloral, chloral betaine, chloral hydrate, chlordiazepoxide, clomipramine, cloperidone, clorazepate, clorethate, clozapine, codeine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydrocodone, hydroxyzine, imipramine, lithium, lorazepam, lormetazepam, maprotaline, mecloqualone, melatonin, mephobarbital, meprobamate, methadone, methaqualone, midaflur, midazolam, nefazodone, nisobamate, nitrazepam, nortriptyline, oxazepam, oxycodone, paraldehyde, paroxetine, pentazocrine, pentobarbital, pergolide, perlapine, perphenazine, phenelzine, phenobarbital, prazepam, promethazine, propofol, propoxyphene, protryptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, and zolpidem, or pharmaceutically acceptable salts thereof. A preferred form of (−)-cis-6-phenyl-5-[4-(2-pyrrolidin-1-yl-ethoxy)-phenyl]-5,6,7,8-tetrahydro-naphthalene-2-ol for use in the pharmaceutical composition is its D-tartrate salt. A preferred second compound for use in the pharmaceutical composition of this invention is sertraline, or its hydrochloride salt.

The present invention also provides kits for use by a consumer to treat joint pain and/or to improve sleep, the kits comprising:

(a) a pharmaceutical composition comprising a SERM as described in any of the first through fourth embodiments, above, and a pharmaceutically acceptable carrier, vehicle or diluent; and

(b) instructions describing a method of using the pharmaceutical composition to treat joint pain and/or to improve sleep.

In a preferred embodiment of the kits, the SERM is (−)-cis-6-phenyl-5-[4-(2-pyrrolidin-1-yl-ethoxy)-phenyl]-5,6,7,8-tetrahydro-naphthalene-2-ol or an optical or geometric isomer thereof; or a pharmaceutically acceptable salt, N-oxide, ester, quaternary ammonium salt or a prodrug thereof.

In another preferred embodiment of the kits, (−)-cis-6-phenyl-5-[4-(2-pyrrolidin-1-yl-ethoxy)-phenyl]-5,6,7,8-tetrahydro-naphthalene-2-ol is a D-tartrate salt.

### DETAILED DESCRIPTION OF THE INVENTION

The terms “treat”, “treatment” and “treating” include preventative (e.g., prophylactic) and palliative treatment or the act of providing preventative or palliative treatment. In the present invention, these terms include the amelioration of joint pain, which means the methods of the present invention are effective in reducing the intensity of the joint pain. The term “pain” is used in the general sense and is meant to encompass pain levels from the merely uncomfortable to the virtually unbearable. The term “joint pain” is used to identify pain in the joint areas of the afflicted patient, such as, but not limited to, pain in the finger, toe, wrist, elbow, shoulder, hip, knee or ankle joints. The term “improving sleep” as used herein means enhancing or improving sleep quality, in particular by increasing sleep efficiency and augmenting sleep maintenance, as well as by preventing and treating sleep disorders and sleep disturbances in a patient. The term “improving sleep” includes the following: an increase in the value which is calculated from the time that a subject sleeps divided by the time that a subject is attempting to sleep; a decrease in sleep latency, i.e., the time it takes to fall asleep; a decrease in difficulties in falling asleep; a decrease in the number of awakenings during sleep; a decrease in nocturnal arousals; a decrease in the time spent awake following the initial onset of sleep; an increase in the total amount of sleep; and increase in the amount and percentage of rapid eye movement (REM) sleep; an increase in the duration and occurrence of REM sleep; a reduction in the fragmentation of REM sleep; an increase in the amount and percentage of stage 2 sleep; an enhancement of EEG-delta activity during sleep; a decrease in the number of awakenings; a decrease in nocturnal arousals, especially early morning awakenings; an increase in daytime alertness; an increased satisfaction with the intensity of sleep; and increased sleep maintenance. Secondary outcomes that may be provided by the present invention include enhanced cognitive function and increased memory retention.

The term “patient” means animals, particularly mammals. Preferred patients are humans.

An “estrogen agonist/antagonist” is a compound that affects some of the same receptors that estrogen does, but not all, and in some instances, it antagonizes or blocks estrogen. Estrogen agonists/antagonists may also be referred to as antiestrogens although they have some estrogenic activity at some estrogen receptors. Estrogen agonists/antagonists are therefore not what are commonly referred to as “pure antiestrogens”. Antiestrogens that can also act as agonists are referred to as Type I antiestrogens. Type I antiestrogens activate the estrogen receptor to bind tightly in the nucleus for a prolonged time but with impaired receptor replenishment (Clark, et al., Steroids 1973;22:707, Capony et al., Mol Cell Endocrinol, 1975;3:233).

The SERMs used in the methods, compositions and kits of the invention may be administered systemically or locally. For systemic use, the SERMs herein are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three or more times daily.

The compounds of formula (I) and formula (IA) used in the methods, compositions and kits of the present invention are described in U.S. Pat. No. 5,552,412. Those compounds are described by the formulae designated herein as formula (I) and (IA) given below:

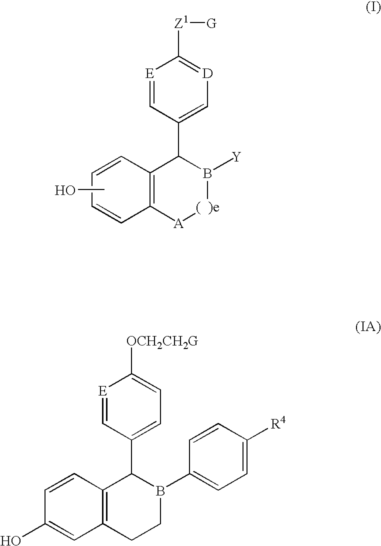

the optical and geometric isomers thereof; and the nontoxic pharmacologically acceptable acid addition salts, N-oxides, esters, quaternary ammonium salts and prodrugs thereof, wherein the variables A, B, D, E, Y, Z¹, G and e are as defined hereinabove for the compound of formula (I) and the variables G, R⁴, B and E are as defined hereinabove for the compound of formula (IA).

Especially preferred compounds used in the methods, compositions and kits of the invention are: cis-6-(4-fluoro-phenyl)-5-[4-(2-piperidin-1-yl-ethoxy)-phenyl]-5,6,7,8-tetrahydro-naphthalene-2-ol; (−)-cis-6-phenyl-5-[4-(2-pyrrolidin-1-yl-ethoxy)-phenyl]-5,6,7,8-tetrahydro-naphthalene-2-ol; cis-6-phenyl-5-[4-(2-pyrrolidin-1-yl-ethoxy)-phenyl]-5,6,7,8-tetrahydro-naphthalene-2-ol; cis-1-[6′-pyrrolidinoethoxy-3′-pyridyl]-2-phenyl-6-hydroxy-1,2,3,4-tetrahydronaphthalene; 1-(4′-pyrrolidinoethoxyphenyl)-2-(4″-fluorophenyl)-6-hydroxy-1,2,3,4-tetrahydroisoquioline; cis-6-(4-hydroxyphenyl)-5-[4-(2-piperidin-1-yl-ethoxy)-phenyl]-5,6,7,8-tetrahydro-naphthalene-2-ol; and 1-(4′-pyrrolidinoethoxyphenyl)-2-phenyl-6-hydroxy-1,2,3,4-tetrahydroisoquinoline and pharmaceutically acceptable salts thereof. An especially preferred salt of (−)-cis-6-phenyl-5-[4-(2-pyrrolidin-1-yl-ethoxy)-phenyl]-5,6,7,8-tetrahydro-naphthalene-2-ol used in the methods, compositions and kits of the present invention is (−)-cis-6-phenyl-5-[4-(2-pyrrolidin-1-yl-ethoxy)-phenyl]-5,6,7,8-tetrahydro-naphthalene-2-ol, D-tartrate.

Other SERMs that can be used in the methods, compositions and kits of the present invention are disclosed in U.S. Pat. No. 5,047,431, including droloxifene.

Additional SERMs that can be used in the methods, compositions and kits of the present invention are tamoxifen: (ethanamine, 2-[-4-(1,2-diphenyl-1-butenyl)phenoxy]-N,N-dimethyl, (Z)-2-, 2-hydroxy-1,2,3-propanetricarboxylate(1:1)) and other compounds as disclosed in U.S. Pat. No. 4,536,516; 4-hydroxy tamoxifen (i.e., tamoxifen wherein the 2-phenyl moiety has a hydroxy group at the 4 position) and other compounds as disclosed in U.S. Pat. No. 4,623,660; raloxifene: (methanone, [6-hydroxy-2-(4-hydroxyphenyl)benzo[b]thien-3-yl][4-[2-(1 -piperidinyl)ethoxy]phenyl]-,hydrochloride) and other compounds as disclosed in U.S. Pat. Nos. 4,418,068; 5,393,763; 5,457,117; 5,478,847 and 5,641,790; toremifene: (ethanamine, 2-[4-(4-chloro-1,2-diphenyl-1-butenyl)phenoxy]-N,N-dimethyl-, (Z)-, 2-hydroxy-1,2,3-propanetricarboxylate (1:1) and other compounds as disclosed in U.S. Pat. Nos. 4,696,949 and 4,996,225; centchroman: 1-[2-[[4-(-methoxy-2,2, dimethyl-3-phenyl-chroman-4-yl)-phenoxy]-ethyl]-pyrrolidine and other compounds as disclosed in U.S. Pat. No. 3,822,287; idoxifene: pyrrolidine, 1-[-[4-[[1-(4-iodophenyl)-2-phenyl-1-butenyl]phenoxy]ethyl] and other compounds as disclosed in U.S. Pat. No. 4,839,155; arzoxifene, 6-hydroxy-3-(4-[2-(piperidin-1-yl)ethoxy]-phenoxy)-2-(4-methoxyphenyl)benzo[b]thiophene hydrochloride, and other compounds as disclosed in U.S. Pat. No. 5,723,474; 6-(4-hydroxy-phenyl)-5-[4-(2-piperidin-1-yl-ethoxy)-benzyl]-naphthalen-2-ol and other compounds as disclosed in U.S. Pat. No. 5,484,795; and {4-[2-(2-aza-bicyclo[2.2.1]hept-2-yl)-ethoxy]-phenyl}-[6-hydroxy-2-(4-hydroxy-phenyl)-benzo[b]thiophen-3-yl]-methanone and other compounds as disclosed in published international patent application WO 95/10513. Other preferred compounds include GW 5638 and GW 7604. The synthesis of these compounds is described in Willson et al., J. Med. Chem., 1994;37:1550-1552.

Further SERMs that can be used in the methods, compositions and kits of the present invention include EM-652 and EM-800. The synthesis of EM-652 and EM-800 and the activity of various enantiomers is described in Gauthier et al., J. Med. Chem., 1997;40:2117-2122. Further SERMs that can be used in the methods of the present invention include bazedoxifene (TSE 424) and other compounds disclosed in U.S. Pat. No. 5,998,402, U.S. Pat. No. 5,985,910, U.S. Pat. No. 5,780,497, U.S. Pat. No. 5,880,137, U.S. Pat. No. 5,998,402, U.S. Pat. No. 6,127,404, U.S. Pat. No. 6,225,308, U.S. Pat. No. 6,232,307, U.S. Pat. No. 6,291,451, U.S. Pat. No. 6,326,367 and U.S. patent application Publication 2001021719.

The expression “pharmaceutically acceptable salts” includes both pharmaceutically acceptable acid addition salts and pharmaceutically acceptable cationic salts. The expression “pharmaceutically-acceptable cationic salts” is intended to define but is not limited to such salts as the alkali metal salts, (e.g. sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to define but is not limited to such salts as the hydrochloride, hydrobromide, sulfate, hydrogen sulfate, phosphate, hydrogen phosphate, dihydrogenphosphate, acetate, succinate, tartrate, citrate, methanesulfonate (mesylate) and p-toluenesulfonate (tosylate) salts.

The SERMs used in the methods, compositions and kits of this invention can be administered in the form of pharmaceutically acceptable salts. The acid-addition salts are conveniently formed, as is usual in organic chemistry, by reacting the compound used in the methods, compositions and kits of this invention with a suitable acid. The salts are quickly formed in high yields at moderate temperatures, and often are prepared by merely isolating the compound from a suitable acidic wash as the final step of the synthesis. The salt-forming acid is dissolved in an appropriate organic solvent, or aqueous organic solvent, such as an alkanol, ketone or ester. A preferred technique for preparing hydrochlorides is to dissolve the free base in a suitable solvent and dry the solution thoroughly, as over molecular sieves, before bubbling hydrogen chloride gas through it. A preferred salt of (−)-cis-6-phenyl-5-[4-(2-pyrrolidin-1-yl-ethoxy)-phenyl]-5,6,7,8-tetrahydro-naphthalene-2-ol is the D-(−)-tartrate salt. On the other hand, if the compound used in the methods, compositions and kits of this invention is desired in the free base form, it is isolated from a basic final wash step, according to the usual practice. Likewise, pharmaceutically-acceptable cationic salts are conveniently formed, as is usual in organic chemistry, by reacting the compound used in the methods, compositions and kits of this invention with a suitable base. The salts are quickly formed in high yields at moderate temperatures, and often are prepared by merely isolating the compound from a suitable basic wash as the final step of the synthesis. The salt-forming base is dissolved in an appropriate organic solvent, or aqueous organic solvent, such as an alkanol, ketone or ester. It will also be recognized that it is possible to administer an amorphous form of a SERM.

One of ordinary skill in the art will recognize that certain SERMs used in the methods, compositions and kits of this invention will contain one or more atoms which may be in a particular stereochemical, tautomeric, or geometric configuration, giving rise to stereoisomers, tautomers and configurational isomers. All such tautomers and isomers and mixtures thereof are included in this invention. Hydrates and solvates of the SERMs used in the methods, compositions and kits of this invention are also included.

The methods, compositions and kits of this invention also include the use of isotopically-labeled SERMs, which are structurally identical to those disclosed above, but for the fact that one or more atoms are replaced by an atom having an atomic mass or mass number different from the atomic mass or mass number usually found in nature. Examples of isotopes that can be incorporated into compounds used in the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Compounds used in the methods, compositions and kits of the present invention, prodrugs thereof, and pharmaceutically acceptable salts of said compounds and of said prodrugs which contain the aforementioned isotopes and/or other isotopes of other atoms are within the scope of this invention. Certain isotopically labeled compounds used in the methods, compositions and kits of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labeled compounds used in the methods, compositions and kits of this invention and prodrugs thereof can generally be prepared by carrying out known or referenced procedures and by substituting a readily available isotopically labeled reagent for a non-isotopically labeled reagent.

Those of ordinary skill in the art will recognize that physiologically active compounds which have accessible hydroxy groups can be administered in the form of pharmaceutically acceptable esters. The compounds used in the methods, compositions and kits of this invention can be effectively administered as an ester, formed on the hydroxy groups, just as one skilled in pharmaceutical chemistry would expect. It is possible, as has long been known in pharmaceutical chemistry, to adjust the rate or duration of action of the compound by appropriate choices of ester groups.

Certain ester groups are preferred when a compound used in the methods, compositions and kits of this invention contains an ester. The SERMs, including the compounds of formula (I) and (IA), may contain ester groups at various positions as defined herein above, where these ester groups are represented as —COOR⁹, in which R⁹ is C₁-C₁₄ alkyl, C₁-C₃ chloroalkyl, C₁-C₃ fluoroalkyl, C₅ -C₇ cycloalkyl, phenyl, or phenyl mono- or disubstituted with C₁-C₄ alkyl, C₁-C₄ alkoxy, hydroxy, nitro, chloro, fluoro or tri(chloro or fluoro)methyl.

As used herein, the term “therapeutically effective amount” means an amount of compound that is capable of treating and/or preventing joint pain or of improving sleep. The specific dose of a compound administered according to this invention will, of course, be determined by the particular circumstances surrounding the case including, for example, the compound administered, the route of administration, the state of being of the patient, the severity of the joint pain being treated or the level of sleep improvement sought. It is intended that a therapeutically effective amount of a second compound is used when a second compound is used in the methods, compositions and kits of this invention.

The magnitude of the prophylactic or therapeutic dose of a compound used in the methods, compositions and kits of this invention to be administered to a patient in the acute or chronic management of joint pain and/or sleep improvement is rather widely variable and subject to the judgement of the attending physician. It should be noted that it may be necessary to adjust the dose of a compound when it is administered in the form of a salt, such as a laureate, the salt forming moiety of which has an appreciable molecular weight.

The following dosage amounts and other dosage amounts set forth elsewhere in this description and in the appendant claims are for an average human subject having a weight of about 65 kg to about 70 kg. The skilled practitioner will readily be able to determine the dosage amount required for a subject whose weight falls outside the 65 kg to 70 kg range, based upon the medical history of the subject. All doses set forth herein, and in the appendant claims, are daily doses of the free base form of the SERMs. Calculation of the dosage amount for other forms of the free base form such as salts or hydrates is easily accomplished by performing a simple ratio relative to the molecular weights of the species involved.

The general range of effective administration rates of a SERM is from about 0.001 mg/day to about 200 mg/day. A preferred rate range is from about 0.010 mg/day to about 100 mg/day. It is often practical to administer the daily dose of compound in portions, at various hours of the day. However, in any given case, the amount of compound administered will depend on such factors as the potency of the specific SERM, the solubility of the compound, the formulation used and the route of administration. When a second compound is used in the methods, compositions and kits of this invention the general range of effective administration rates of the second compound is from about 0.001 mg/day to about 2000 mg/day. However, in any given case, the amount of the second compound administered will depend on such factors as the potency of the specific second compound, the solubility of the compound, the formulation used and the route of administration.

Methods of formulation are well known in the art and are disclosed, for example, in Remington's Pharmaceutical Sciences, Mack Publishing Company, Easton, Pa., 19th Edition (1995). Pharmaceutical compositions for use within the present invention can be in the form of sterile, non-pyrogenic liquid solutions or suspensions, coated capsules, suppositories, lyophilized powders, transdermal patches or other forms known in the art.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant may be necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances that facilitate the disintegration of a tablet to release a compound when the tablet becomes wet. They include starches, clays, celluloses, algins and gums, more particularly, corn and potato starches, methylcellulose, agar, bentonite, wood cellulose, powdered natural sponge, cation-exchange resins, alginic acid, guar gum, citrus pulp and carboxymethylcellulose, for example, may be used as well as sodium lauryl sulfate.

Tablets are often coated with sugar as a flavorant and sealant, or with film- forming protecting agents to modify the dissolution properties of the tablet. The compounds may also be formulated as chewable tablets, by using large amounts of pleasant-tasting substances such as mannitol in the formulation, as is now well- established in the art.

When it is desired to administer a compound as a suppository, the typical bases may be used. Cocoa butter is a traditional suppository base, which may be modified by addition of waxes to raise its melting point slightly. Water-miscible suppository bases comprising, particularly, polyethylene glycols of various molecular weights are in wide use.

The topical formulation can be in the form of a cream, jelly, ointment, gel, lotion, paste or for application by a patch. The topical preparation can be administered to the skin at or in the vicinity of the joint pain.

The effect of the compounds may be delayed or prolonged by proper formulation. For example, a slowly soluble pellet of the compound may be prepared and incorporated in a tablet or capsule. The technique may be improved by making pellets of several different dissolution rates and filling capsules with a mixture of the pellets. Tablets or capsules may be coated with a film which resists dissolution for a predictable, period of time. Topical, formulations may be designed to yield delayed and/or prolonged percutaneous absorption of a compound. Even the parenteral preparations may be made long-acting, by dissolving or suspending the compound in oily or emulsified vehicles which allow it to disperse only slowly in the serum.

The term “prodrug” means a compound that is transformed in vivo to yield a compound used in the methods, compositions and kits of the present invention. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound used in the methods, compositions and kits of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N, N-(C₁-C₂)alkylamino(C₂-C₃)alkyl (such as N,N-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound used in the methods, compositions and kits of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-i-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a compound of the present invention comprises an amine functional group, a prodrug can be formed by the replacement of a hydrogen atom in the amine group with a group such as R^{X}-carbonyl, R^{X}O-carbonyl, NR^{X}R^{X}′-carbonyl where R^{X} and R^{X}′ are each independently (C₁-C₁₀)alkyl, (C₃-C₇)cycloalkyl, benzyl, or R^{X}-carbonyl is a natural α-aminoacyl or natural α-aminoacyl-natural α-aminoacyl, —C(OH)C(O)OY^{X} wherein Y^{X} is H, (C₁-C₆)alkyl or benzyl), —C(OY^{X0}) Y^{X1} wherein Y^{X0} is (C₁-C₄) alkyl and Y^{X1} is (C₁-C₆)alkyl, carboxy(C₁-C₆)alkyl, amino(C₁-C₄)alkyl or mono-N— or di-N,N—(C₁-C₆)alkylaminoalkyl, —C(^{Y2}) Y^{X3} wherein Y^{X2} is H or methyl and Y^{X3} is mono-N— or di-N,N—(C₁-C₆)alkylamino, morpholino, piperidin-1-yl or pyrrolidin-1-yl.

Advantageously, the present invention also provides kits for use by a consumer to treat joint pain and/or improve sleep. The kits comprise a) a pharmaceutical composition comprising a SERM and a pharmaceutically acceptable carrier, vehicle or diluent; and b) instructions describing a method of using the pharmaceutical compositions to treat joint pain and/or improve sleep. The instructions may also indicate that the kit is to treat joint pain and/or improve sleep while substantially reducing the concomitant liability of adverse effects associated with estrogen administration.

A “kit” as used in the instant application includes a container for containing the pharmaceutical compositions and may also include divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or patient, e.g., in the form of numbers next to the tablets or capsules whereby the numbers correspond with the days of the regimen which the tablets or capsules so specified should be ingested or a card which contains the same type of information. Another example of such a memory aid is a calendar printed on the card e.g., as follows “First Week, Monday, Tuesday,” . . . etc . . . . “Second Week, Monday, Tuesday, . . . ” etc. Other variations of memory aids will be readily apparent. A “daily dose” can be a single tablet or capsule or several tablets or capsules to be taken on a given day.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The kits of the present invention may also include, in addition to a SERM, one or more additional pharmaceutically active compounds. Preferably, the additional compound is another SERM or another compound useful to treat joint pain or to improve sleep. The additional compounds may be administered in the same dosage form as the SERM or in different dosage forms. Likewise, the additional compounds can be administered at the same time as the SERM or at different times.

Compounds that are used to treat and/or prevent joint pain and which can be used in combination with the SERMs in the methods, compositions and kits of the present invention include nonsteroidal anti-inflammatory drugs (NSAIDs), such as aspirin, acetominophen, ibuprofen, naproxen, ketoprofen, nabumetone, etodolac, salsalate, sulindac, diclofenac, tolmetin, flurbiprofen, piroxicam, fenoprofen, indomethacin, meclofenamate, oxaprozin, diflunisal and ketorolac; and selective cyclooxygenase-2 (COX-2) inhibitors such as rofecoxib (Vioxx®), celecoxib (Celebrex®), valdecoxib (Bextra®) and etoricoxib. NSAIDs can have unwanted side effects such as ulcers, therefore NSAIDs are sometimes administered with other compounds that ameliorate the side effects. Typical compounds that are used in combination with NSAIDs include proton pump inhibitors such as omeprazole; antacids such as sucralfate; and H2 blockers such as ranitidine, cimetidine, famotidine and nizatidine. Thus, the combination aspect of the present invention comprises a SERM, an NSAID and a compound that reduces a side effect of an NSAID. In addition, the combination aspect of the present invention also includes the coadministration of a SERM and a COX-2 inhibitor. For example, (−)-cis-6-phenyl-5-[4-(2-pyrrolidin-1-yl-ethoxy)-phenyl]-5,6,7,8-tetrahydro-naphthalene-2-ol or an optical or geometric isomer thereof; a pharmaceutically acceptable salt, N-oxide, ester, quaternary ammonium salt, or a prodrug thereof can be administered with 4-[5-(4-methylphenyl)-3-(trifluoromethyl)-1H-pyrazol-1-yl]benzenesulfonamide (celecoxib), 4-[4-(methylsulfonyl)phenyl]-3-phenyl-2(5H)-furanone (rofecoxib), valdecoxib or etoricoxib. A preferred salt of (−)-cis-6-phenyl-5-[4-(2-pyrrolidin-1-yl-ethoxy)-phenyl]-5,6,7,8-tetrahydro-naphthalene-2-ol is the D-tartrate. The coadministration can be in the same dosage form or different dosage forms and at the same time or at different times. All possible modes and schedules of administration are contemplated. In addition, products derived from natural substances have been used to treat joint pain. Examples include hyaluronic acid, glucosamine, chondroitin sulfate and capsaicin. Corticosteriods have also been used to treat joint pain. The second compound from the compounds that are used to treat and/or prevent joint pain can be administered at a dosage that is therapeutically effective for the treatment of joint pain. All of the above compounds and others that can be used to treat joint pain can be used in combination with the SERMs used in the methods, compositions and kits of the present invention.

Compounds that are used to improve sleep and which can also be used in combination with the SERMs in the methods, compositions and kits of the present invention include adinazolam, allobarbital, alonimid, alprazolam, amitryptiline, amobarbital, amoxapine, bentazepam, benzoctamine, bromocriptine, brotizolam, bupropion, buspirone, butabarbital, butalbital, capuride, carbocloral, chloral betaine, chloral hydrate, chlordiazepoxide, clomipramine, cloperidone, clorazepate, clorethate, clozapine, codeine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydrocodone, hydroxyzine, imipramine, lithium, lorazepam, lormetazepam, maprotaline, mecloqualone, melatonin, mephobarbital, meprobamate, methadone, methaqualone, midaflur, midazolam, nefazodone, nisobamate, nitrazepam, nortriptyline, oxazepam, oxycodone, paraldehyde, paroxetine, pentazocrine, pentobarbital, pergolide, perlapine, perphenazine, pheneizine, phenobarbital, prazepam, promethazine, propofol, propoxyphene, protryptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem and pharmaceutically acceptable salts thereof. The second compound from the compounds that are used to improve sleep can be administered at a dosage that is therapeutically effective for the improvement of sleep. All of the above compounds and others that can be used to improve sleep can be used in combination with the SERMs used in the methods, compositions and kits of the present invention.

It is noted that the SERMs can be administered in the same dosage form (e.g., a tablet) or in different dosage forms. The compounds can be administered at the same time or at different times. All such variations are intended to be encompassed by the combination aspect of the present invention.

The effects of a SERM on joint pain can be determined by administering a compound to a patient having, or at risk of having, joint pain for a time and observing the results. Likewise, the effects of a SERM on improving sleep can be determined by administering a compound to a patient in need of sleep improvement for a time and observing the results. All documents cited herein, including patents and patent applications, are hereby incorporated by reference.

### EXAMPLE 1

The Effects of Oral Administration of (−)-cis-6-phenyl-5-[4-(2-pyrrolidin-1-yl-ethoxy)-phenyl]-5,6,7,8-tetrahydro-naphthalene-2-ol, D-tartrate salt (Iasofoxifene) on Joint Pain: A Randomized, Double Blind, Placebo Controlled Study

Subjects: A double blind, randomized, placebo controlled trial of approximately three hundred and seventy five women between the ages of 50 and 74 years of age inclusive, and 1-20 years postmenopausal was carried out. The subjects selected for the study were ambulatory females between the ages of 50 and 74 years of age (inclusive) who gave written informed consent for participation in the study. The subjects' last menstrual period or episode of vaginal bleeding was at least one year, but not more than 20 years prior to the initial screening visit and their estradiol level was <110 pmol/L (30 pg/ml) and follicle-stimulating hormone (FSH)>30 IU/L. Minor deviations from these postmenopausal criteria, in subjects who are clearly postmenopausal, could be accepted on a case-by-case basis. The subjects selected for the study had a body mass index less than 32 with the body mass index being defined as the weight in kilograms divided by the height in meters squared. In all cases, however, the subject's weight was less than or equal to 94 kg and height less than 72 inches (183 cm). The subject's Screening/Baseline mean L1-L4 lumbar bone mineral density (BMD) of the spine must be less than +2.0 standard deviations and greater than −2.5 standard deviations of age-and sex-matched bone mineral density (Z-score) as determined by standards of the manufacturer of the Dual X-ray Absorptiometer (DXA) used in the determination of that BMD. The subjects' CBC with differential and platelet count; urinalysis; and Multichem 23 or equivalent (calcium, inorganic phosphorus, sodium, potassium, chloride, lactic acid dehydroenase (LDH), serum glutamic oxaloacetic transaminase (SGOT (AST)), serum glutamic pyruvic transaminase (SGPT (ALT)), total bilirubin, alkaline phosphatase, total protein, globulin, albumin, blood urea nitrogen (BUN), creatinine, uric acid, glucose) were within +/−10% of the limits of the upper and lower range of normal. Serum thyroid stimulating hormone (TSH) will be between 0.4 and 6.0 mIU/ml, inclusive (Subjects on a fixed dose of thyroid hormone for six months or more, with TSH values within the specified range, are eligible for inclusion). The subjects' resting 12-lead electrocardiogram were within clinically normal limits and the subjects' gynecological examination including Papanicolaou (Pap) smear were normal. Minor abnormalities in cervical cytology, e.g. minor atypia or inflammation, were not grounds for exclusion. The subjects' endometrial thickness were typically <5 mm as determined by transvaginal ultrasound (TVU) and subjects outside of this criteria were asked to undergo additional baseline evaluation following initial randomization. The subjects had a mammogram within the 3 months prior to screening examination that showed neither cancer nor suspicion of cancer warranting biopsy or, if a mammogram had not been obtained within the previous 3 months, subjects had one obtained as part of the screening procedure.

Women with a current history of severe or disabling postmenopausal symptoms (i.e. hot flushes) that would warrant open label estrogen therapy were excluded from the study. Women with a history of medical disease that might be associated with the development of metabolic bone disease including: bone marrow disease; hereditary disorders of calcium or mineral metabolism; Paget's Disease; untreated or inadequately treated endocrine disorders including untreated thyrotoxicosis, hyper- or hypoparathyroidism, adrenal disorders, and insulin-dependent diabetes mellitus; rheumatoid arthritis or other connective tissue disorders; gastrointestinal diseases including chronic liver disease, partial or total resection of the stomach or bowel, or malabsorption syndrome were also excluded from the study.

Women with a history of other significant medical disorders currently requiring chronic medical therapy that may interfere with the conduct of the study (disorders such as any degree of renal insufficiency, poorly controlled hypertension or that requiring more than 2 agents for control, unstable angina, or subjects with history of myocardial infarction within the previous 6 months) were excluded from the study. Women with a history of malignancy within the previous five years with the exception of basal cell carcinoma, curatively treated by surgery and/or localized gynecological cancer treated by total hysterectomy were excluded from the study.

Women who had undergone therapy with an investigational drug during the last 30 days preceding admission to the study or administration of: estrogen or estrogenic compounds (including hormonal estrogen agonistantagonists such as tamoxifen, raloxifene, idoxifene, levormeloxifene, toremifene, phytoestrogens, or DHEA and. similar agents) within the 3 months prior to screening visit, calcitonin or related products within the 3 months prior to screening, or sodium fluoride (at doses >2 mg/day) or any of the bisphosphonates (e.g. didronel or alendronate) within the 12 months prior to screening visit were also excluded from the study.

Subjects taking any of the following medications within the previous 12 months: anabolic steroids, chronic glucocorticoid or related steroids, glutethimide, heparin, coumadin, anticonvulsants of any kind, doses of vitamin D above 800 units daily, doses of calcium above 1500 mg daily, sodium fluoride (above 2 mg/day), inhaled steriods for asthma or chronic obstructive pulmonary disease (COPD) were excluded from the study. Women who smoke more than 10 cigarettes per day or consume more than 2 units per day of alcohol were excluded from the study. A unit of alcohol is defined as 2 ounces of hard liquor, 4 ounces of wine, or 12 ounces of beer. Women with a personal history of recurrent superficial phlebitis, deep venous thrombosis, pulmonary embolus, retinal vein thrombosis, or greater than 6 months use of anticoagulants at any time in their past or women with a strong family history of recurrent deep vein thrombosis (DVT) or pulmonary embolism were also excluded from the study.

Women with scoliosis or other clinical spinal deformity severe enough to invalidate lumbar bone densitometry in the L1-L4 region were excluded from the study. Other such conditions include spinal fusion surgery, previous traumatic fracture of the spine, or aortic calcifications severe enough to invalidate bone mineral density assessment in the lumbar region. Evaluation for such exclusions may be based on clinical history, spine DXA evaluation, or plain radiographs of the spine. Women with a history of nontraumatic fractures of the vertebrae, or recurrent fractures of other bones such as the wrist, foot, rib or pelvis, that suggests the presence of underlying metabolic bone disease other than normal postmenopausal bone loss or a fracture of either hip were excluded from the study. Subjects determined by transvaginal ultrasound (TVU) to have significant ovarian pathology (simple cysts >2 cm or complex cysts of any size) or uterine pathology (endometrial thickness >5 mm and/or hyperplasia/cancer).

Patients were randomly allocated to one of four treatment regimen groups (Groups A, B, C, D) using a computer generated pseudo-random code generated using the method of random permutated blocks. These patients received (in a double blind fashion):

Group A (n=75) placebo

Group B (n=75) 0.25 mg/day of (−)-cis-6-phenyl-5-[4-(2-pyrrolidin-1-yl-ethoxy)-phenyl]-5,6,7,8-tetrahydro-naphthalene-2-ol, D-tartrate salt

Group C (n=75) 1.0 mg/day of (−)-cis-6-phenyl-5-[4-(2-pyrrolidin-1-yl-ethoxy)-phenyl]-5,6,7,8-tetrahydro-naphthalene-2-ol, D-tartrate salt

Group D (n=150) 60.0 mg/day of raloxifene

Each patient also received two tablets daily, each tablet containing calcium 500 mg and vitamin D 125 IU.

The placebo, 0.25 mg/day and 1.0 mg/day of (−)-cis-6-phenyl-5-[4-(2-pyrrolidin-1-yl-ethoxy)-phenyl]-5,6,7,8-tetrahydro-naphthalene-2-ol, D-tartrate salt (lasofoxifene) and raloxifene tablets were dispensed in blister pack cards. The patients were instructed to take the medication in the morning or if the patient had difficulty in complying with morning dosing then to take the medication at bedtime or with the evening meal. Neither the investigator or patient was aware of the constituents of the tablets provided in the blister packs.

Assessment Method: Patients were asked to record their pain score using a standardized linear visual analog scale (VAS; 100 mm scale, 0 mm=no pain, 100 mm=worst pain). The patients were asked to provide the pain score at 3 months, 6 months, 12 months, 18 months and 24 months of the study.

Result: The results of the 3 months assessment for joint pain in the 0.25 lasofoxifene, 1.0 mg lasofoxifene and placebo treatment groups are provided in Table 1 below. Adjusted Analysis of Variance (ANCOVA) technique was used to examine the effects of the various treatments on self-reported joint pain at the 3 months assessment. At the 3 months assessment the lasofoxifene 1.0 mg treatment group reported a mean change from baseline of 4.52 for joint pain whereas the placebo treatment group reported a mean change from baseline of 5.26 for joint pain. At the 3 months assessment the lasofoxifene 0.25 mg treatment group reported a mean change from baseline of −8.16 for joint pain whereas the placebo treatment group reported a mean change from baseline of 5.26 for joint pain. These mean change scores from baseline revealed a statistically significant (p<0.001) reduction in self-reported joint pain in the lasofoxifene 0.25 mg treatment group compared to the placebo treatment group at the 3 months assessment.

TABLE 1

| | Joint Pain Changes Resulting | Reduction in |
|---|---|---|
| | From Treatment | Joint Pain |
| Treatment Group | (Mean Change from Baseline) | Compared to Placebo |
| Lasofoxifene | −8.16 | P = 0.0012 |
| 0.25 mg | | |
| Lasofoxifene | 4.52 | P = 0.9902 |
| 1.0 mg | | |
| Placebo | 5.26 | 0 |

### EXAMPLE 2

The Effects of Oral Administration of (−)-cis-6-phenyl-5-[4-(2-pyrrolidin-1-yl-ethoxy)-phenyl]-5,6,7,8-tetrahydro-naphthalene-2-ol, D-tartrate salt (lasofoxifene) on Sleep: A Randomized, Double Blind, Placebo Controlled Study

Subjects: A double blind, randomized, placebo controlled trial of approximately three hundred and seventy five women between the ages of 50 and 74 years of age inclusive, and 1-20 years postmenopausal was carried out. The subjects selected for the study were ambulatory females between the ages of 50 and 74 years of age (inclusive) who gave written informed consent for participation in the study. The subjects' last menstrual period or episode of vaginal bleeding was at least one year, but not more than 20 years prior to the initial screening visit and their estradiol level was <110 pmol/L (30 pg/ml) and FSH>30 IU/L. Minor deviations from these postmenopausal criteria, in subjects who are clearly postmenopausal, could be accepted on a case-by-case basis. The subjects selected for the study had a body mass index less than 32 with the body mass index being defined as the weight in kilograms divided by the height in meters squared. In all cases, however, the subject's weight was less than or equal to 94 kg and height less than 72 inches (183 cm). The subject's Screening/Baseline mean L1-L4 lumbar bone mineral density (BMD) of the spine must be less than +2.0 standard deviations and greater than −2.5 standard deviations of age-and sex-matched bone mineral density (Z-score) as determined by standards of the manufacturer of the Dual X-ray Absorptiometer (DXA) used in the determination of that BMD. The subjects' CBC with differential and platelet count; urinalysis; and Multichem 23 or equivalent (calcium, inorganic phosphorus, sodium, potassium, chloride, LDH, SGOT (AST), SGPT (ALT), total bilirubin, alkaline phosphatase, total protein, globulin, albumin, BUN, creatinine, uric acid, glucose) were within +/− 10% of the limits of the upper and lower range of normal. Serum thyroid stimulating hormone (TSH) will be between 0.4 and 6.0 mIU/ml, inclusive (Subjects on a fixed dose of thyroid hormone for six months or more, with TSH values within the specified range, are eligible for inclusion). The subjects' resting 12-lead electrocardiogram were within clinically normal limits and the subjects' gynecological examination including Papanicolaou (Pap) smear were normal. Minor abnormalities in cervical cytology, e.g. minor atypia or inflammation, were not grounds for exclusion. The subjects' endometrial thickness were typically <5 mm as determined by transvaginal ultrasound (TVU) and subjects outside of this criteria were asked to undergo additional baseline evaluation following initial randomization. The subjects had a mammogram within the 3 months prior to screening examination that showed neither cancer nor suspicion of cancer warranting biopsy or, if a mammogram had not been obtained within the previous 3 months, subjects had one obtained as part of the screening procedure.

Women with a current history of severe or disabling postmenopausal symptoms (i.e. hot flushes) that would warrant open label estrogen therapy were excluded from the study. Women with a history of medical disease that might be associated with the development of metabolic bone disease including: bone marrow disease; hereditary disorders of calcium or mineral metabolism; Paget's Disease; untreated or inadequately treated endocrine disorders including untreated thyrotoxicosis, hyper- or hypoparathyroidism, adrenal disorders, and insulin-dependent diabetes mellitus; rheumatoid arthritis or other connective tissue disorders; gastrointestinal diseases including chronic liver disease, partial or total resection of the stomach or bowel, or malabsorption syndrome were also excluded from the study.

Women with a history of other significant medical disorders currently requiring chronic medical therapy that may interfere with the conduct of the study (disorders such as any degree of renal insufficiency, poorly controlled hypertension or that requiring more than 2 agents for control, unstable angina, or subjects with history of myocardial infarction within the previous 6 months) were excluded from the study. Women with a history of malignancy within the previous five years with the exception of basal cell carcinoma, curatively treated by surgery and/or localized gynecological cancer treated by total hysterectomy were excluded from the study.

Women who had undergone therapy with an investigational drug during the last 30 days preceding admission to the study or administration of: estrogen or estrogenic compounds (including hormonal estrogen agonist/antagonists such as tamoxifen, raloxifene, idoxifene, levormeloxifene, toremifene, phytoestrogens, or DHEA and similar agents) within the 3 months prior to screening visit, calcitonin or related products within the 3 months prior to screening, or sodium fluoride (at doses >2 mg/day) or any of the bisphosphonates (e.g. didronel or alendronate) within the 12 months prior to screening visit were also excluded from the study.

Subjects taking any of the following medications within the previous 12 months: anabolic steroids, chronic glucocorticoid or related steroids, glutethimide, heparin, coumadin, anticonvulsants of any kind, doses of vitamin D above 800 units daily, doses of calcium above 1500 mg daily, sodium fluoride (above 2 mg/day), inhaled steriods for asthma or COPD were excluded from the study. Women who smoke more than 10 cigarettes per day or consume more than 2 units per day of alcohol were excluded from the study. A unit of alcohol is defined as 2 ounces of hard liquor, 4 ounces of wine, or 12 ounces of beer. Women with a personal history of recurrent superficial phlebitis, deep venous thrombosis, pulmonary embolus, retinal vein thrombosis, or greater than 6 months use of anticoagulants at any time in their past or women with a strong family history of recurrent deep vein thrombosis (DVT) or pulmonary embolism were also excluded from the study.

Women with scoliosis or other clinical spinal deformity severe enough to invalidate lumbar bone densitometry in the L1-L4 region were excluded from the study. Other such conditions include spinal fusion surgery, previous traumatic fracture of the spine, or aortic calcifications severe enough to invalidate bone mineral density assessment in the lumbar region. Evaluation for such exclusions may be based on clinical history, spine DXA evaluation, or plain radiographs of the spine. Women with a history of nontraumatic fractures of the vertebrae, or recurrent fractures of other bones such as the wrist, foot, rib or pelvis, that suggests the presence of underlying metabolic bone disease other than normal postmenopausal bone loss or a fracture of either hip were excluded from the study. Subjects determined by transvaginal ultrasound (TVU) to have significant ovarian pathology (simple cysts >2 cm or complex cysts of any size) or uterine pathology (endometrial thickness ≧5 mm and/or hyperplasia/cancer).

Patients were randomly allocated to one of four treatment regimen groups (Groups A, B, C, D, E) using a computer generated pseudo-random code generated using the method of random permutated blocks. These patients received (in a double blind fashion):

Group A (n=50) placebo

Group B (n=50) 0.40 mg/day of (−)-cis-6-phenyl-5-[4-(2-pyrrolidin-1-yl-ethoxy)-phenyl]-5,6,7,8-tetrahydro-naphthalene-2-ol, D-tartrate salt

Group C (n=50) 1.25 mg/day of (−)-cis-6-phenyl-5-[4-(2-pyrrolidin-1-yl-ethoxy)-phenyl]-5,6,7,8-tetrahydro-naphthalene-2-ol, D-tartrate salt

Group D (n=50) 5.0 mg/day of (−)-cis-6-phenyl-5-[4-(2-pyrrolidin-1-yl-ethoxy)-phenyl]-5,6,7,8-tetrahydro-naphthalene-2-ol, D-tartrate salt

Group E (n=50) 0.625 mg/2.5 mg of conjugated estrogens/medroxyprogesterone (Prem Pro®))

Each patient also received two tablets daily, each tablet containing calcium 500 mg and vitamin D 125 IU.

The placebo, 0.4 mg/day, 1.25 mg/day and 5.0 mg/day of (−)-cis-6-phenyl-5-[4-(2-pyrrolidin- 1-yl-ethoxy)-phenyl]-5,6,7,8-tetrahydro-naphthalene-2-ol, D-tartrate salt (lasofoxifene) and 0.625 mg/2.5 mg of conjugated estrogens/medroxyprogesterone (PremPro®)) tablets were dispensed in blister pack cards. The patients were instructed to take the medication in the morning or if the patient had difficulty in complying with morning dosing then to take the medication at bedtime or with the evening meal. Neither the investigator or patient was aware of the constituents of the tablets provided in the blister packs.

### The effects of Iasofoxifene on Sleep

Assessment Method: Patient self-report recorded on Sleep Problems subscale of standardized questionnaire, Women's Health Questionnaire (WHQ; Hunter, M. 1992. Psychology and Health, 7: 45-54).

Result: in adjusted between group t-tests, the difference in the mean change scores from baseline revealed a statistically significant improvement in sleep (ie, less sleep disturbance) in the lasofoxifene 0.4mg treatment group vs. placebo at 3 months assessment.

TABLE 2

| | Mean Change | Difference |
|---|---|---|
| Treatment Grp | from Baseline | from Placebo |
| Lasofoxifene 0.4 mg | −0.09 | P = 0.05 |
| Placebo | 0.03 | 0 |

What is claimed is:

1. A method of treating joint pain or improving sleep in a patient, the method comprising administering to said patient in need thereof a therapeutically effective amount of a SERM of formula (I): 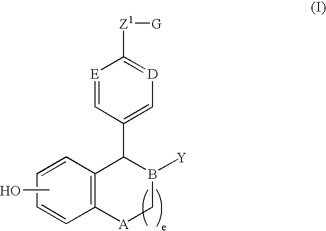
wherein:
A is selected from CH₂ and NR;
B, D and E are independently selected from CH and N;
Y is
(a) phenyl, optionally substituted with 1-3 substituents independently selected from R⁴;
(b) naphthyl, optionally substituted with 1-3 substituents independently selected from R⁴;
(c) C₃-C₈ cycloalkyl, optionally substituted with 1-2 substituents independently selected from R⁴;
(d) C₃-C₈ cycloalkenyl, optionally substituted with 1-2 substituents independently selected from R⁴;
(e) a five membered heterocycle containing up to two heteroatoms selected from the group consisting of —O—, —NR²— and —S(O)ₙ—, optionally substituted with 1-3 substituents independently selected from R⁴;
(f) a six membered heterocycle containing up to two heteroatoms selected from the group consisting of —O—, —NR²— and —S(O)ₙ— optionally substituted with 1-3 substituents independently selected from R⁴; or
(g) a bicyclic ring system consisting of a five or six membered heterocyclic ring fused to a phenyl ring, said heterocyclic ring containing up to two heteroatoms selected from the group consisting of —O—, —NR²— and —S(O)ₙ—, optionally substituted with 1-3 substituents independently selected from R⁴;
Z¹ is
(a) —(CH₂)ₚW(CH₂)_{q}—;
(b) —O(CH₂)ₚCR⁵R⁶—;
(c) —O(CH₂)ₚW(CH₂)_{q}—;
(d) —OCHR²CHR³—; or
(e) —SCHR²CHR³—;
G is
(a) —NR⁷R⁸; 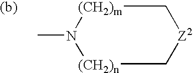
wherein n is 0, 1 or 2; m is 1, 2 or 3; Z² is —NH—, —O—, —S—, or —CH₂—; optionally fused on adjacent carbon atoms with one or two phenyl rings and, optionally independently substituted on carbon with one to three substituents and, optionally, independently on nitrogen with a chemically suitable substituent selected from R⁴; or
(c) a bicyclic amine containing five to twelve carbon atoms, either bridged or fused and optionally substituted with 1-3 substituents independently selected from R⁴; or
Z¹ and G in combination may be 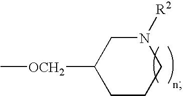
W is
(a) —CH₂—;
(b) —CH=CH—;
(c) —O—;
(d) —NR²;
(e) —S(O)ₙ—; 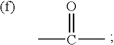
(g) —CR²(OH)—;
(h) —CONR²—;
(i) —NR²CO—; 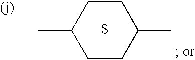
(k) —C≡C—;
R is hydrogen or C₁-C₆ alkyl;
R² and R³ are independently
(a) hydrogen; or
(b) C₁-C₄ alkyl;
R⁴ is
(a) hydrogen;
(b) halogen;
(c) C₁-C₆ alkyl;
(d) C₁-C₄ alkoxy;
(e) C₁-C₄ acyloxy;
(f) C₁-C₄ alkylthio;
(g) C₁-C₄ alkylsulfinyl;
(h) C₁-C₄ alkylsulfonyl;
(i) hydroxy (C₁-C₄)alkyl;
aryl (C₁-C₄)alkyl;
(k) —CO₂H;
(l) —CN;
(m) —CONHOR;
(n) —SO₂NHR;
(o) —NH₂;
(p) C₁-C₄ alkylamino;
(q) C₁-C₄ dialkylamino;
(r) —NHSO₂R;
(s) —NO₂;
(t) -aryl; or
(u) —OH;
R⁵ and R⁶ are independently C₁-C₈ alkyl or together form a C₃-C₁₀ carbocyclic ring;
R⁷ and R⁸ are independently
(a) phenyl;
(b) a C₃-C₁₀ carbocyclic ring, saturated or unsaturated;
(c) a C₃-C₁₀ heterocyclic ring containing up to two heteroatoms, selected from —O—, —N— and —S—;
(d) H;
(e) C₁-C₆ alkyl; or
(f) form a 3 to 8 membered nitrogen containing ring with R⁵ or R⁶.
R⁷ and R⁸ in either linear or ring form may optionally be substituted with up to three substituents independently selected from C₁-C₆ alkyl, halogen, alkoxy, hydroxy and carboxy;
a ring formed by R⁷ and R⁸ may be optionally fused to a phenyl ring;
e is 0, 1 or 2;
m is 1, 2 or 3;
n is 0, 1 or 2;
p is 0, 1, 2 or 3;
q is 0, 1, 2 or 3;
or an optical or geometric isomer thereof; or a pharmaceutically acceptable salt, N-oxide, ester, quaternary ammonium salt or prodrug thereof; and provided that said joint pain is not joint pain resulting from osteoarthritis or rheumatoid arthritis.

2. The method of claim 1 wherein the SERM is a compound of formula (IA) 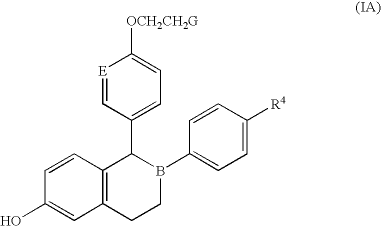
wherein G is 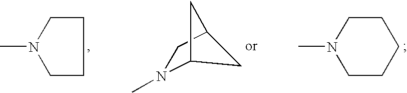
R⁴ is H, OH, F, or Cl; and B and E are independently selected from CH and N or an optical or geometric isomer thereof; or a pharmaceutically acceptable salt, N-oxide, ester, quaternary ammonium salt, or a prodrug thereof.

3. The method of claim 1 wherein the SERM is (−)-cis-6-phenyl-5-[4-(2-pyrrolidin-1-yl-ethoxy)-phenyl]-5,6,7,8-tetrahydro-naphthalene-2-ol or an optical or geometric isomer thereof; a pharmaceutically acceptable salt, N-oxide, ester, quaternary ammonium salt, or a prodrug thereof.

4. The method of claim 3 wherein the SERM is (−)-cis-6-phenyl-5-[4-(2-pyrrolidin-1-yl-ethoxy)-phenyl]-5,6,7,8-tetrahydro-naphthalene-2-ol, D-tartrate salt.

5. The method of any one of claims 1-4 wherein the method is the method of treating joint pain.

6. The method of any one of claims 1-4 wherein the method is the method of improving sleep.

7. A method of treating joint pain in a patient, the method comprising administering to said patient in need thereof a therapeutically effective amount of a first compound, said first compound being a SERM of formula (I): 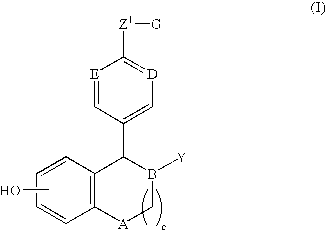
wherein:
A is selected from CH₂ and NR;
B, D and E are independently selected from CH and N;
Y is
(a) phenyl, optionally substituted with 1-3 substituents independently selected from R⁴;
(b) naphthyl, optionally substituted with 1-3 substituents independently selected from R⁴;
(c) C₃-C₈ cycloalkyl, optionally substituted with 1-2 substituents independently selected from R⁴;
(d) C₃-C₈ cycloalkenyl, optionally substituted with 1-2 substituents independently selected from R⁴;
(e) a five membered heterocycle containing up to two heteroatoms selected from the group consisting of —O—, —NR²— and —S(O)ₙ—, optionally substituted with 1-3 substituents independently selected from R⁴;
(f) a six membered heterocycle containing up to two heteroatoms selected from the group consisting of —O—, —NR²— and —S(O)ₙ— optionally substituted with 1-3 substituents independently selected from R⁴; or
(g) a bicyclic ring system consisting of a five or six membered heterocyclic ring fused to a phenyl ring, said heterocyclic ring containing up to two heteroatoms selected from the group consisting of —O—, —NR²— and —S(O)ₙ—, optionally substituted with 1-3 substituents independently selected from R⁴;
Z′ is
(a) —(CH₂)ₚ W(CH₂)_{q}—;
(b) —O(CH₂)ₚ CR⁵R⁶—;
(c) —O(CH₂)ₚW(CH₂)_{q}—;
(d) —OCHR²CHR³—; or
(e) —SCHR²CHR³—;
G is
(a) —NR⁷R⁸; 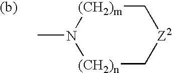
wherein n is 0, 1 or 2; m is 1, 2 or 3; Z² is —NH—, —O—, —S—, or —CH₂—; optionally fused on adjacent carbon atoms with one or two phenyl rings and, optionally independently substituted on carbon with one to three substituents and, optionally, independently on nitrogen with a chemically suitable substituent selected from R⁴; or
(c) a bicyclic amine containing five to twelve carbon atoms, either bridged or fused and optionally substituted with 1-3 substituents independently selected from R⁴; or
Z¹ and G in combination may be 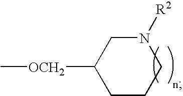
W is
(a) —CH₂—;
(b) —CH═CH—;
(c) —O—;
(d) —NR²—;
(e) —S(O)ₙ—; 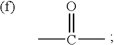
(g) —CR²(OH)—;
(h) —CONR²—;
(i) —NR²CO—; 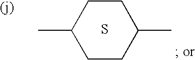
(k) —C≡C—;
R is hydrogen or C₁-C₆ alkyl;
R² and R³ are independently
(a) hydrogen; or
(b) C₁-C₄ alkyl;
R⁴ is
(a) hydrogen;
(b) halogen;
(c) C₁-C₆ alkyl;
(d) C₁-C₄ alkoxy;
(e) C₁-C₄ acyloxy;
(f) C₁-C₄ alkylthio;
(g) C₁-C₄ alkylsulfinyl;
(h) C₁-C₄ alkylsulfonyl;
(i) hydroxy (C₁-C₄)alkyl;
(j) aryl (C₁-C₄)alkyl;
(k) —CO₂H;
(l) —CN;
(m) —CONHOR;
(n) —SO₂NHR;
(o) —NH₂;
(p) C₁-C₄ alkylamino;
(q) C₁-C₄ dialkylamino;
(r) —NHSO₂R;
(s) —NO₂;
(t) -aryl; or
(u) —OH;
R⁵ and R⁶ are independently C₁-C₈ alkyl or together form a C₃-C₁₀ carbocyclic ring;
R⁷ and R⁸ are independently
(a) phenyl;
(b) a C₃-C₁₀ carbocyclic ring, saturated or unsaturated;
(c) a C₃-C₁₀ heterocyclic ring containing up to two heteroatoms, selected from —O—, —N— and —S—;
(d) H;
(e) C₁-C₆ alkyl; or
(f) form a 3 to 8 membered nitrogen containing ring with R⁵ or R⁶;
R⁷ and R⁸ in either linear or ring form may optionally be substituted with up to three substituents independently selected-from C₁-C₆ alkyl, halogen, alkoxy, hydroxy and carboxy;
a ring formed by R⁷ and R⁸ may be optionally fused to a phenyl ring;
e is 0, 1 or 2;
m is 1, 2 or 3;
n is 0, 1 or 2;
p is 0, 1, 2 or 3;
q is 0, 1, 2 or 3;
or an optical or geometric isomer thereof; or a pharmaceutically acceptable salt, N-oxide, ester, quaternary ammonium salt or prodrug thereof; and
a therapeutically effective amount of a second compound, said second compound selected from the group consisting of acetominophen, aspirin, ibuprofen, naproxen, ketoprofen, nabumetone, etodolac, salsalate, sulindac, diclofenac, tolmetin, flurbiprofen, piroxicam, fenoprofen, indomethacin, meclofenamate, oxaprozin, diflunisal, ketorolac, celecoxib, rofecoxib, valdecoxib, etoricoxib, hyaluronic acid, glucosamine, chondroitin and capsaicin; provided that said joint pain is not joint pain resulting from osteoarthritis or rheumatoid arthritis.

8. The method of claim 7 wherein the first compound is (−)-cis-6-phenyl-5-[4-(2-pyrrolidin-1-yl-ethoxy)-phenyl]-5,6,7,8-tetrahydro-naphthalene-2-ol, D-tartrate salt.

9. The method of claim 8 wherein the second compound is celecoxib, rofecoxib, valdecoxib or etoricoxib.

10. A method of improving sleep in a patient, the method comprising administering to said patient in need thereof a therapeutically effective amount of a first compound, said first compound being a SERM of formula (I): 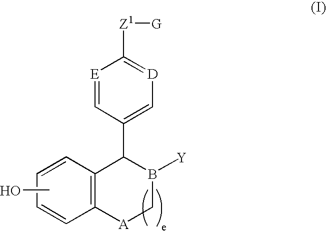
wherein:
A is selected from CH₂ and NR;
B, D and E are independently selected from CH and N;
Y is
(a) phenyl, optionally substituted with 1-3 substituents independently selected from R⁴;
(b) naphthyl, optionally substituted with 1-3 substituents independently selected from R⁴;
(c) C₃-C₈ cycloalkyl, optionally substituted with 1-2 substituents independently selected from R⁴;
(d) C₃-C₈ cycloalkenyl, optionally substituted with 1-2 substituents independently selected from R⁴;
(e) a five membered heterocycle containing up to two heteroatoms selected from the group consisting of —O—, —NR²— and —S(O)ₙ—, optionally substituted with 1-3 substituents independently selected from R⁴;
(f) a six membered heterocycle containing up to two heteroatoms selected from the group consisting of —O—, —NR²— and —S(O)ₙ— optionally substituted with 1-3 substituents independently selected from R⁴; or
(g) a bicyclic ring system consisting of a five or six membered heterocyclic ring fused to a phenyl ring, said heterocyclic ring containing up to two heteroatoms selected from the group consisting of —O—, —NR²— and —S(O)ₙ—, optionally substituted with 1-3 substituents independently selected from R⁴;
Z¹ is
(a) —(CH₂)ₚ W(CH₂)_{q}—;
(b) —O(CH₂)ₚ CR⁵R⁶—;
(c) —O(CH₂)ₚW(CH₂)_{q}—;
(d) —OCHR²CHR³—; or
(e) —SCHR²CHR³—;
G is
(a) NR⁷R⁸; 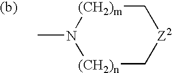
wherein n is 0, 1 or 2; m is 1, 2 or 3; Z² is —NH—, —O—, —S—, or —CH₂—; optionally fused on adjacent carbon atoms with one or two phenyl rings and, optionally independently substituted on carbon with one to three substituents and, optionally, independently on nitrogen with a chemically suitable substituent selected from R⁴; or
(c) a bicyclic amine containing five to twelve carbon atoms, either bridged or fused and optionally substituted with 1-3 substituents independently selected from R⁴; or
Z¹ and G in combination may be 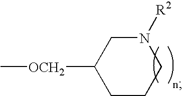
W is
(a) —CH₂—;
(b) —CH═CH—;
(c) —O—;
(d) —NR²—;
(e) —S(O)ₙ—; 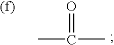
(g) —CR²(OH)—;
(h) —CONR²—;
(i) —NR²CO—; 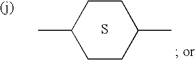
(k) —C≡C—;
R is hydrogen or C₁-C₆ alkyl;
R² and R³ are independently
(a) hydrogen; or
(b) C₁-C₄ alkyl;
R⁴ is
(a) hydrogen;
(b) halogen;
(c) C₁-C₆ alkyl;
(d) C₁-C₄ alkoxy;
(e) C₁-C₄ acyloxy;
(f) C₁-C₄ alkylthio;
(g) C₁-C₄ alkylsulfinyl;
(h) C₁-C₄ alkylsulfonyl;
(i) hydroxy (C₁-C₄)alkyl;
(j) aryl (C₁-C₄)alkyl;
(k) —CO₂H;
(l) —CN;
(m) —CONHOR;
(n) —SO₂NHR;
(o) —NH₂;
(p) C₁-C₄ alkylamino;
(q) C₁-C₄ dialkylamino;
(r) —NHSO₂R;
(s) —NO₂;
(t) -aryl; or
(u) —OH;
R⁵ and R⁶ are independently C₁-C₈ alkyl or together form a C₃-C₁₀ carbocyclic ring;
R⁷ and R⁸ are independently
(a) phenyl;
(b) a C₃-C₁₀ carbocyclic ring, saturated or unsaturated;
(c) a C₃-C₁₀ heterocyclic ring containing up to two heteroatoms, selected from —O—, —N— and —S—;
(d) H;
(e) C₁-C₆ alkyl; or
(f) form a 3 to 8 membered nitrogen containing ring with R⁵ or R⁶;
R⁷ and R⁸ in either linear or ring form may optionally be substituted with up to three substituents independently selected from C₁-C₆ alkyl, halogen, alkoxy, hydroxy and carboxy;
a ring formed by R⁷ and R⁸ may be optionally fused to a phenyl ring;
e is 0, 1 or 2;
m is 1, 2 or 3;
n is 0, 1 or 2;
p is 0, 2 or 3;
q is 0, 1, 2 or 3;
or an optical or geometric isomer thereof; or a pharmaceutically acceptable salt, N-oxide, ester, quaternary ammonium salt or prodrug thereof; and a therapeutically effective amount of a second compound, said second compound being selected from the group consisting of adinazolam, allobarbital, alonimid, alprazolam, amitryptiline, amobarbital, amoxapine, bentazepam, benzoctamine, bromocriptine, brotizolam, bupropion, buspirone, butabarbital, butalbital, capuride, carbocloral, chloral betaine, chloral hydrate, chlordiazepoxide, clomipramine, cloperidone, clorazepate, clorethate, clozapine, codeine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydrocodone, hydroxyzine, imipramine, lithium, lorazepam, lormetazepam, maprotaline, mecloqualone, melatonin, mephobarbital, meprobamate, methadone, methaqualone, midaflur, midazolam, nefazodone, nisobamate, nitrazepam, nortriptyline, oxazepam, oxycodone, paraldehyde, paroxetine, pentazocrine, pentobarbital, pergolide, perlapine, perphenazine, phenelzine, phenobarbital, prazepam, promethazine, propofol, propoxyphene, protryptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and pharmaceutically acceptable salts thereof.

11. The method of claim 10 wherein the first compound is (−)-cis-6-phenyl-5-[4-(2-pyrrolidin-1-yl-ethoxy)-phenyl]-5,6,7,8-tetrahydro-naphthalene-2-ol, D-tartrate salt.

12. The method of claim 11 wherein the second compound is sertraline.

13. A pharmaceutical composition comprising: (−)-cis-6-phenyl-5-[4-(2-pyrrolidin-1-yl-ethoxy)-phenyl]-5,6,7,8-tetrahydro-naphthalene-2-ol or a pharmaceutically acceptable salt thereof; and a second compound selected from the group consisting of adinazolam, allobarbital, alonimid, alprazolam, amitryptiline, amobarbital, amoxapine, bentazepam, benzoctamine, bromocriptine, brotizolam, bupropion, buspirone, butabarbital, butalbital, capuride, carbocloral, chloral betaine, chloral hydrate, chlordiazepoxide, clomipramine, cloperidone, clorazepate, clorethate, clozapine, codeine, cyprazepam, desipramine, dexciamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydrocodone, hydroxyzine, imipramine, lithium, lorazepam, lormetazepam, maprotaline, mecloqualone, melatonin, mephobarbital, meprobamate, methadone, methaqualone, midaflur, midazolam, nefazodone, nisobamate, nitrazepam, nortriptyline, oxazepam, oxycodone, paraldehyde, paroxetine, pentazocrine, pentobarbital, pergolide, perlapine, perphenazine, pheneizine, phenobarbital, prazepam, promethazine, propofol, propoxyphene, protryptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, and zolpidem, or pharmaceutically acceptable salts thereof.

14. The pharmaceutical composition of claim 13 wherein the (−)-cis-6-phenyl-5-[4-(2-pyrrolidin-1-yl-ethoxy)-phenyl]-5,6,7,8-tetrahydro-naphthalene-2-ol is in the form of the D-tartrate salt.

15. The pharmaceutical composition of claim 14 wherein the second compound is sertraline, or a pharmaceutically acceptable salt thereof.
